(19) United States
(12) Patent Application Publication
(10) Pub. No.: US 2007/0148113 A1
(43) Pub. Date: Jun. 28, 2007

(54) Antiperspirant compositions comprising at least one dispersion of cationic colloidal silica particles, antiperspirant product, and cosmetic process for treating perspiration

(76) Inventors: Cyril Lemoine, Saint Cyr L'Ecole (FR); Philippe Ilekti, Maison-Alfort (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/633,441

(22) Filed: Dec. 5, 2006

Related U.S. Application Data
(60) Provisional application No. 60/757,848, filed on Jan. 11, 2006.

(30) Foreign Application Priority Data
Dec. 5, 2005 (FR) 05 53718

(51) Int. Cl. A61K 8/28; A61K 8/26

(52) U.S. Cl. 424/65; 424/66; 424/68

(57) ABSTRACT

The present disclosure relates to the use of a dispersion of cationic colloidal particles of silica modified with a multivalent cation in a cosmetic composition, as an antiperspirant active agent. The present disclosure also relates to antiperspirant compositions comprising at least one dispersion of cationic colloidal particles of silica modified with a multivalent cation, as well as cosmetic processes for treating perspiration and body odor, employing the compositions disclosed herein.

This application claims benefit of U.S. Provisional Application Ser. No. 60/757,848, filed Jan. 11, 2006, the contents of which are incorporated herein by reference. This application also claims benefit of priority under 35 U.S.C. § 119 to French Patent Application No. FR 05 53718, filed Dec. 5, 2005, the contents of which are also incorporated herein by reference.

The present disclosure relates to the use of a dispersion of cationic colloidal particles of silica modified with a multivalent cation, as an antiperspirant active agent in a cosmetic composition for reducing perspiration.

In cosmetics, it is well-known to use a topical application of antiperspirant products comprising substances that have the effect of limiting or even suppressing the flow of sweat. These products are generally available in the form of roll-ons, sticks, aerosols and sprays.

Antiperspirant substances generally comprise aluminum salts or aluminum/zirconium salts. The use of these active agents at high concentrations in order to obtain good efficacy may result in formulation difficulties. Furthermore, these substances may lead to irritation of the skin.

Thus, there is a need to find novel antiperspirant active agents that can replace aluminum salts and aluminum/zirconium salts, and that are effective and easy to formulate.

The inventors have discovered, surprisingly, that a dispersion of cationic colloidal particles of silica modified with a multivalent cation in a physiologically acceptable medium constitutes a good antiperspirant agent and can be readily formulated in numerous products for reducing perspiration, without requiring the use of standard astringent salts.

Accordingly, one aspect of the present disclosure is the use of a dispersion of cationic colloidal particles of silica modified with a multivalent cation in a cosmetic composition.

Another aspect of the present disclosure relates to antiperspirant compositions comprising at least one dispersion of colloidal particles of silica modified with a multivalent cation wherein the dispersion is present in an amount effective to reduce human perspiration.

The present disclosure also relates to a cosmetic process for treating perspiration, comprising applying to the surface of the skin an effective amount of a dispersion of cationic colloidal particles of silica modified with a multivalent cation.

As used herein, the term “antiperspirant agent” is understood to mean any substance which, by itself, has the effect of reducing or limiting the flow of sweat.

As used herein, the term “antiperspirant composition” is understood to mean any composition which, when applied to the surface of the skin, has the effect of reducing or limiting the flow of sweat.

As used herein, the term “divalent cation” is understood to mean a cation having a valency of greater than or equal to 2, for instance, according to at least one embodiment, the valency is equal to 3.

For the purposes of the present disclosure, the term “dispersion of colloidal silica particles” is understood to mean a dispersion of silica (SiO₂) particles with a number-average diameter ranging from 3 to 150 nm, for example ranging from 5 to 30 nm, and further, for example, ranging from 10 to 25 nm. These particles conserve the above-mentioned diameters in the composition comprising them, without substantially aggregating, and they do not have any thickening properties, in the sense that at a concentration of greater than or equal to 15% by weight in water, the colloidal particles according to the present disclosure have a viscosity of less than 0.05 Pa.s for a shear rate equal to 10⁻¹s, the viscosity being measured at 25° C. using a RheoStress RS150 rheometer from Haake in cone-plate configuration, the cone having a diameter of 60 mm and an angle of 20. According to at least one embodiment, the medium for dispersing the inorganic colloidal particles is chosen from an aqueous, aqueous-alcoholic or alcoholic medium. For instance, according to at least one embodiment, the medium is an aqueous medium.

For the purposes of the present disclosure, the term “cationic particles of silica modified with a multivalent cation” is understood to mean particles comprising silicon dioxide and whose surface has been chemically modified with cations forming at least one monomolecular layer of the cation; each cation being linked to the silica so as to make it cationic. Such composite particles generally comprise from 5% to 60% by weight, for example, from 10% to 50% by weight, of silica (SiO₂) and the proportion of their surface covered with multivalent cations can range from 1% to 100%, for instance from 2% to 60% and further still, for example, from 5% to 60%.

The cation present in the colloidal particles of modified silica may be chosen from alkaline-earth metals, for instance calcium (Ca²⁺), magnesium (Mg²⁺), strontium (Sr²⁺) or barium (Ba²⁺); transition metals, for instance titanium (Ti²⁺, Ti³⁺, Ti⁴⁺), manganese (Mn²⁺, Mn³⁺, Mn⁴⁺, Mn⁷⁺), iron (Fe²⁺, Fe³⁺), zirconium (Zr⁴⁺), hafnium (Hf4+) or (Al³⁺).

According to at least one embodiment, the cation is chosen from aluminum (Al³⁺) and/or zirconium (Zr⁴⁺) and/or hafnium (Hf4+). For instance, according to at least one embodiment the cation is aluminum (Al³⁺).

The colloidal particles of cationically modified silica may also additionally comprise a stabilizing anionic counterion, such as chloride (Cl⁻), for example.

Among the dispersions of cationic colloidal particles of silica modified with aluminum Al³⁺ in accordance with the present disclosure, non-limiting mention may be made of those commercially available from the company Grace under the trade reference Ludox® such as, for example, the product Ludox CL® (25% silica (SiO₂) with a mean particle size of about 12 nm and stabilized with Cl⁻ions) of structure:

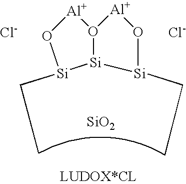

According to at least one embodiment, the cationic colloidal silica particles in accordance with the present disclosure used as antiperspirant active agents are present in the compositions according to the present disclosure in amounts ranging from 1% to 80%, for instance ranging from 5% to 50% and further for example from 10% to 50% by weight of active material relative to the total weight of the composition.

The composition according to the present disclosure has a pH of less than or equal to 7. According to at least one embodiment, the composition has a pH ranging from 3 to 6.5.

The composition according to the present disclosure may be in any galenical form conventionally used for topical application, for instance in the form of aqueous gels or aqueous or aqueous-alcoholic solutions. By addition of a fatty or oily phase, it may also be in the form of dispersions of the lotion type, of emulsions of liquid or semi-liquid consistency of the milk type, obtained by dispersing a fatty phase in an aqueous phase (O/W) or conversely (W/O), or of suspensions or emulsions of soft, semi-solid or solid consistency of the cream or gel type, or alternatively multiple emulsions (W/O/W or O/W/O), microemulsions, vesicular dispersions of ionic and/or nonionic type, or wax/aqueous phase dispersions. These compositions are prepared according to the usual methods.

According to at least one aspect of the present disclosure, the antiperspirant compositions are conditioned such that they can be contained in an aerosol device or a pump-dispenser bottle; in a device equipped with a perforated wall such as a grille; in the form of wands (sticks) wherein they comprise at least one dispersion of colloidal particles of silica modified with a cation as defined above. In this regard, they contain the ingredients generally used in products of this type, which are well-known to those skilled in the art.

The compositions according to the present disclosure intended for cosmetic use may comprise at least one aqueous phase. For instance, they may be formulated as aqueous lotions or as water-in-oil, oil-in-water emulsions, or as multiple emulsions (oil-in-water-in-oil or water-in-oil-in-water triple emulsions (such emulsions are known and described, for example, by C. Fox in “Cosmetics and Toiletries”, November 1986, Vol. 101, pages 101-112)).

The aqueous phase of the compositions can comprise water, and water with at least one other water-soluble or water-miscible solvent. The water-soluble or water-miscible solvents may include short-chain monoalcohols, for example of C₁-C₄, for instance ethanol or isopropanol; diols or polyols, for instance ethylene glycol, 1,2-propylene glycol, 1,3-butylene glycol, hexylene glycol, diethylene glycol, dipropylene glycol, 2-ethoxyethanol, diethylene glycol monomethyl ether, triethylene glycol monomethyl ether and sorbitol. According to at least one embodiment, propylene glycol and glycerol will be used.

The compositions according to the present disclosure may comprise at least one water-immiscible organic liquid phase. This phase generally comprises at least one hydrophobic compound that renders the phase water-immiscible. The phase is liquid (in the absence of a structuring agent) at room temperature (20-25° C.). According to at least one embodiment as disclosed herein, the water-immiscible organic liquid phase can comprise at least one oil and comprises at least 80% of compounds with a vapor pressure not exceeding 4 kPa (30 mmHg) at 25° C.

For instance, the water-immiscible organic liquid phase may comprise at least one oil chosen from volatile and non-volatile, silicone-based and hydrocarbon-based emollient oil. The at least one emollient oil is described in U.S. Pat. Nos. 4,822,596 and 4,904,463.

Volatile silicones are defined, in a known manner, as being compounds that are volatile at room temperature. Among these compounds, non-limiting mention may be made of cyclic and linear volatile silicones of the dimethylsiloxane type whose chains comprise from 3 to 9 silicone-based residues. According to at least one embodiment, the volatile silicones may be chosen from cyclomethicones D₄, D₅ or D₆.

Non-volatile silicones are defined, in a known manner, as being compounds with a low vapor pressure at room temperature. The following are included among these compounds, by means of non-limiting example: polyalkylsiloxanes, such as linear polyalkylsiloxanes, for instance the linear polydimethylsiloxanes, or dimethicones, sold by the company Dow Corning under the name “Dow Corning 245 Fluid”; polyalkylarylsiloxanes, for instance the polymethylphenylsiloxanes sold by the company Dow Corning under the name “Dow Corning 556 Fluid”; copolymers of polyether and siloxane, for instance dimethicone copolyols.

Among the non-volatile emollient oils that may be used in the present disclosure, examples that may be mentioned include but are not limited to: hydrocarbon-based derivatives, mineral oils, fatty alcohols, esters of C₃-C₁₈ alcohols with C₃-C₁₈ acids, esters of benzoic acid with C₁₂-C₁₈ alcohols and mixtures thereof, C₂-C₆ polyols for instance those chosen from glycerol, propylene glycol and sorbitol, and polyalkylene glycol polymers.

The emollient oils may be present in an amount ranging from 1% to 50% by weight, for example, from 5% to 40% by weight relative to the total weight of the composition.

According to another embodiment, the antiperspirant composition comprises at least one dispersion of cationic colloidal particles of silica modified with a cation as defined above and at least one additional antiperspirant active agent.

The optional at least one additional antiperspirant active agent may be chosen from aluminum and/or zirconium salts; complexes of zirconium hydroxychloride and of aluminum hydroxychloride with an amino acid, such as those described in U.S. Pat. No. 3,792,068, commonly known as “ZAG complexes”. Such complexes are generally known under the name “ZAG” when the amino acid is glycine. The ZAG complexes ordinarily have an Al/Zr quotient ranging from about 1.67 to 12.5 and a metal/Cl quotient ranging from about 0.73 to 1.93. Among these products, non-limiting mention may be made of aluminum zirconium octachlorohydrex GLY, aluminum zirconium pentachlorohydrex GLY, aluminum zirconium tetrachlorohydrate GLY and aluminum zirconium trichlorohydrate GLY.

The aluminum salts that may be mentioned include but are not limited to: aluminum chlorohydrate, aluminum chlorohydrex, aluminum chlorohydrex PEG, aluminum chlorohydrex PG, aluminum dichlorohydrate, aluminum dichlorohydrex PEG, aluminum dichlorohydrex PG, aluminum sesquichlorohydrate, aluminum sesquichlorohydrex PEG, aluminum sesquichlorohydrex PG, alum salts, aluminum sulfate, aluminum zirconium octachlorohydrate, aluminum zirconium pentachlorohydrate, aluminum zirconium tetrachlorohydrate, aluminum zirconium trichlorohydrate, such as the aluminum hydroxychloride sold by the company Reheis under the name Reach 301 or by the company Guilini Chemie under the name Aloxicoll PF 40. Aluminum zirconium salts are, for example, the salt sold by the company Reheis under the name Reach AZP-908-SUF.

The at least one additional antiperspirant active agent may be present in the composition according to the present disclosure in a proportion of about 0.001 % to 30% by weight and according to at least one embodiment, for instance, the additional antiperspirant agent is present in a proportion of about 0.1% to 25% by weight relative to the total composition.

The cosmetic compositions according to the present disclosure may also optionally comprise at least one deodorant active agent, for instance, non-limiting mention may be made of:
bacteriostatic agents or bactericidal agents such as 2,4,4′-trichloro-2′-hydroxydiphenyl ether (Triclosan), 2,4-dichloro-2′-hydroxydiphenyl ether, 3′,4′,5′-trichlorosalicylanilide, 1-(3′,4′-dichlorophenyl)-3-(4′-chlorophenyl)urea (Triclocarban) or 3,7,11-trimethyldodeca-2,5,10-trienol (Farnesol); quaternary ammonium salts, for instance cetyltrimethylammonium salts or cetylpyridinium salts; chlorhexidine and salts; diglyceryl monocaprate, diglyceryl monolaurate or glyceryl monolaurate; polyhexamethylene biguanide salts, or mixtures thereof;
zinc salts, for instance zinc salicylate, zinc phenolsulfonate, zinc pyrrolidonecarboxylate (more commonly known as zinc pidolate), zinc sulfate, zinc chloride, zinc lactate, zinc gluconate, zinc ricinoleate, zinc glycinate, zinc carbonate, zinc citrate, zinc chloride, zinc laurate, zinc oleate, zinc orthophosphate, zinc stearate, zinc tartrate, zinc lactate and zinc acetate, or mixtures thereof.

In order to improve the antiperspirant efficacy of the composition, at least one water-soluble anionic polymers comprising a Bronsted acid, such as those derived from maleic acid and/or from maleic anhydride, which are described in European Patent Application WO 02/49590, may also be used.

In order to improve the homogeneity of the product, it is also possible to use at least one suspension agent, for instance those chosen from hydrophobic-modified montmorillonite clays, for instance hydrophobic-modified bentonites or hectorites. Examples that may be mentioned in a non-limiting manner include but are not limited to the product stearalkonium bentonite (CTFA name) (product of reaction of bentonite and the quaternary ammonium stearalkonium chloride), such as the commercial product sold under the name Tixogel MP 250 by the company Sud Chemie Rheologicals, United Catalysts Inc. or the product disteardimonium hectorite (CTFA name) (product of reaction of hectorite and of distearyldimonium chloride) sold under the name Bentone 38 or Bentone Gel by the company Elementis Specialities.

The at least one suspension agent may be present in an amount ranging from 0.1% to 5% by weight relative to the total weight of the composition. According to at least one embodiment, the at least one suspension agent is present in an amount ranging from 0.2% to 2% by weight relative to the total weight of the composition.

The composition according to the present disclosure may also contain at least one filler.

Among the fillers that may be used according to the present disclosure, non-limiting mention may be made of organic powders. As disclosed herein, the term “organic powder” is understood to mean any solid that is insoluble in the medium at room temperature (25° C.).

Among organic powders that may be used in the composition of the present disclosure, examples that may be mentioned in a non-limiting manner include polyamide particles, for instance those sold under the name Orgasol by the company Atochem; polyethylene powders; microspheres based on acrylic copolymers, such as those made of ethylene glycol dimethacrylate/lauryl methacrylate copolymer, sold by the company Dow Corning under the name Polytrap; polymethyl methacrylate microspheres, sold under the name Microsphere M-100 by the company Matsumoto or under the name Covabead LH85 by the company Wackherr; ethylene-acrylate copolymer powders, for instance those sold under the name Flobeads by the company Sumitomo Seika Chemicals; expanded powders such as hollow microspheres, for instance microspheres formed from a terpolymer of vinylidene chloride, of acrylonitrile and of methacrylate and sold under the name Expancel by the company Kemanord Plast under the references 551 DE 12 (particle size of about 12 pm and density of 40 kg/m³), 551 DE 20 (particle size of about 30 μm and density of 65 kg/m³) and 551 DE 50 (particle size of about 40 μm), or the microspheres sold under the name Micropearl F 80 ED by the company Matsumoto; powders of natural organic materials such as starch powders, for instance corn starch, wheat starch and rice starch, which may or may not be crosslinked, such as the starch powder crosslinked with octenylsuccinate anhydride, sold under the name Dry-Flo by the company National Starch; silicone resin microbeads such as those sold under the name Tospearl by the company Toshiba Silicone, such as Tospearl 240; amino acid powders such as the lauroyllysine powder sold under the name Amihope LL-11 by the company Ajinomoto; particles of wax microdispersion, for example, those that have mean sizes of less than 1 μm, for instance those ranging from 0.02 μm to 1 μm, and which comprise at least one wax, such as the products sold under the name Aquacer by the company Byk Cera, including by means of non-limiting example: Aquacer 520 (mixture of synthetic and natural waxes), Aquacer 514 or 513 (polyethylene wax), Aquacer 511 (polymer wax), or such as the products sold under the name Jonwax 120 by the company Johnson Polymer (mixture of polyethylene wax and paraffin wax) and under the name Ceraflour 961 by the company Byk Cera (micronized modified polyethylene wax); and mixtures thereof.

The cosmetic compositions according to the present disclosure may also comprise at least one cosmetic adjuvant chosen from waxes, softeners, antioxidants, opacifiers, stabilizers, moisturizers, vitamins, fragrances, bactericides, preserving agents, polymers, fragrances, thickeners, propellants and any other ingredient usually used in cosmetics for this type of application.

Needless to say, a person skilled in the art will take care to select the optional additional compound(s) such that the beneficial properties intrinsically associated with the cosmetic composition in accordance with the present disclosure are not, or are not substantially, adversely affected by the envisaged addition(s).

The waxes may be chosen from animal, fossil, plant, mineral and synthetic waxes. Non-limiting mention may be made for instance of beeswaxes, carnauba wax, candelilla wax, sugar cane wax, Japan wax, ozokerites, montan wax, microcrystalline waxes, paraffins and silicone waxes and resins.

The thickeners, according to at least one embodiment are nonionic, and may be chosen from modified or unmodified guar gums and celluloses, such as hydroxypropyl guar gum or cetylhydroxyethylcellulose, silicas, for instance Bentone Gel MIO sold by the company NL Industries, or Veegum Ultra sold by the company Polyplastic.

The thickeners may also be cationic, for instance Polyquaternium-37 sold under the name Salcare SC95 (Polyquaternium-37 (and) Mineral Oil (and) PPG-1 Trideceth-6) or Salcare SC96 (Polyquaternium-37 (and) propylene glycol dicaprylate/dicaprate (and) PPG-1-Trideceth-6) or other crosslinked cationic polymers, for instance those of CTFA name ethyl acrylate/dimethylaminoethyl methacrylate cationic copolymer in emulsion.

The amounts of these various constituents that may be present in the cosmetic composition according to the present disclosure are those conventionally used in antiperspirant compositions.

When they contain a water-immiscible organic liquid phase, the compositions according to the present disclosure may also comprise at least one agent for structuring or gelling the water-immiscible organic liquid phase of the composition, such as linear solid fatty alcohols and/or waxes; fatty acids or salts thereof (stearic acid, sodium stearate or 12-hydroxystearic acid); dibenzylidene alditols (DBS); lanosterol, N-acylamino acid derivatives; di- or tricarboxylic acid derivatives, for instance alkyl-N,N′-dialkylsuccin-amides (i.e.: dodecyl-N,N′-dibutylsuccinamide); elastomeric polyorganosiloxanes such as those described in European Patent Application No. WO 97/44010.

The compositions according to the present disclosure may also be pressurized and may be packaged in an aerosol device comprising:
(a) a container comprising an antiperspirant composition as defined above, and
(b) at least one propellant and a device for distributing said composition.

The at least one propellant generally used in products of this type, which are well-known to those skilled in the art, are, for example, dimethyl ether (DME); volatile hydrocarbons such as n-butane, propane or isobutane, and mixtures thereof, optionally with at least one chlorohydrocarbon and/or fluorohydrocarbon; among the latter, non-limiting mention may be made of the compounds sold by the company Dupont de Nemours under the names Freon® and Dymel®, for example, monofluorotrichloromethane, difluorodichloromethane, tetrafluorodichloroethane and 1,1-difluoroethane, for instance sold under the trade name Dymel 152 A by the company Dupont. Carbon dioxide, nitrous oxide, nitrogen and compressed air may also be used as the at least one propellant.

The compositions comprising the at least one antiperspirant active polymer as defined above and the at least one propellant may be in the same compartment or in different compartments in the aerosol container. According to the present disclosure, the concentration of the at least one propellant generally ranges from 5% to 95% by pressurized weight, for example, ranging from 50% to 85% by weight relative to the total weight of the pressurized composition.

The distribution device, which forms a part of the aerosol device, may comprise a distribution valve controlled by a distribution head, itself comprising a nozzle via which the aerosol composition is vaporized. The container comprising the pressurized composition may be opaque or transparent. For instance, it may be made of glass, of polymeric material or of metal, optionally coated with a coat of protective varnish.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the disclosure are approximations, the numerical values set forth in the specific example is reported as precisely as possible. Any numerical value, however, inherently contains certain errors necessarily resulting from the standard deviation found in its respective testing measurement.

The example that follows is intended to illustrate the present disclosure without, however, being limiting in nature.

### EXAMPLE

The antiperspirant efficacy of an aqueous dispersion containing 20% active material of cationic colloidal particles of silica modified with aluminum (Al³⁺), with a mean size of about 12 nm stabilized with Cl, at a pH of about 4, of the type Ludox CL sold by Grace, was evaluated.

This antiperspirant efficacy was compared:
with that of an aqueous dispersion containing 20% active material of anionic colloidal silica (of particle size 18 nm, Cosmo S 40) sold by the company Catalysts and Chemicals,
with that of a standard aluminum chlorohydrate, Chlorhydrol 50% USP sold by the company Reheis and formulated in an aqueous-alcoholic roll-on containing 10% by weight of active material.

### PROTOCOL

The tests were performed on 22 individuals.

Two sets of eight areas (4×5 cm²) were delimited on either side of the backbone. For each product area there was a corresponding symmetrical untreated control area.

The colloidal dispersion of silica-alumina was applied for 4 days at a rate of 75 mg or 75 μl to each corresponding product area. An occlusion was applied for 1 hour. The individuals remained throughout the occlusion period in a room maintained at 30° C. and 50% relative humidity.

Twenty-four hours after the final application, the back was washed with water to remove any trace of remaining product; cellulose squares were attached to the various areas and the individuals were made to sweat in a sauna for 15 minutes at 80° C.

The amount of sweat was evaluated by weighing the cellulose squares before and after sweating. The percentage reduction of sweat relative to the control area without product was determined. A negative reduction percentage indicated that the amount of sweat collected was higher than that in the product-free control area.

A significant positive reduction in the percentage of perspiration, of 16% relative to the product-free control area, was obtained with the colloidal particle dispersion in accordance with the present disclosure. This indicated that the dispersion had significant antiperspirant activity. A standard antiperspirant roll-on of the aqueous-alcoholic type comprising 15% by weight of aluminum chlorohydrate active material gave a positive reduction in the percentage of perspiration of 11%.

On the other hand, with the aqueous dispersion at pH 4 containing 20% active material of Cosmo S40 colloidal particles, an absence of antiperspirant efficacy, or even a 13% increase in perspiration, was obtained.

What is claimed is:

1. An antiperspirant composition comprising:
at least one dispersion of cationic colloidal particles of silica modified with a multivalent cation, wherein the dispersion is present in an amount effective to reduce human perspiration.

2. The antiperspirant composition according to claim 1, wherein the silica particles have a number-average diameter ranging from 3 to 150 nm.

3. The antiperspirant composition according to claim 2, wherein the silica particles have a number-average diameter ranging from 10 to 25 nm.

4. The antiperspirant composition according to claim 1, wherein the silica particles comprise from 5% to 60% by weight of silica (SiO₂).

5. The antiperspirant composition according to claim 4, wherein the silica particles generally comprise from 10% to 50% by weight of silica (SiO₂).

6. The antiperspirant composition according to claim 1, wherein the at least one dispersion of cationic colloidal particles of silica modified with a multivalent cation is in a dispersion medium chosen from an aqueous, aqueous-alcoholic or alcoholic medium.

7. The antiperspirant composition according to claim 6, wherein the dispersion medium is an aqueous medium.

8. The antiperspirant composition according to claim 1, wherein the silica particles have a proportion of cation-covered surface ranging from 1% to 100%.

9. The antiperspirant composition according to claim 8, wherein the silica particles have a proportion of cation-covered surface ranging from 20% to 40%.

10. The antiperspirant composition according to claim 1, wherein the cation present in the colloidal particles of modified silica is chosen from alkaline-earth metals, transition metals and aluminum (Al³⁺).

11. The antiperspirant composition according to claim 10, wherein the cation is chosen from aluminum (Al³⁺) and/or zirconium (Zr⁴⁺) and/or hafnium (Hf4+).

12. The antiperspirant composition according to claim 11, wherein the cation is aluminum (Al³⁺).

13. The antiperspirant composition according to claim 1, wherein the modified silica particles further comprise an anionic stabilizing counterion.

14. The antiperspirant composition according to claim 13, wherein the stabilizing counterion is chloride (Cl⁻).

15. The antiperspirant composition according to claim 1, wherein the cationic colloidal silica particles are present in an amount ranging from 1% to 80% by weight of active material, relative to the total weight of the composition.

16. The antiperspirant composition according to claim 15, wherein the cationic colloidal silica particles are present in an amount ranging from 10% to 50% by weight of active material, relative to the total weight of the composition.

17. The antiperspirant composition according to claim 1, wherein the pH is less than or equal to 7.

18. The antiperspirant composition according to claim 17, wherein the pH ranges from 3 to 6.5.

19. The antiperspirant composition according to claim 1, wherein it is conditioned such that it can be contained in an aerosol device, a pump-dispenser bottle, a roll-on, a device equipped with a perforated wall, or a wand (stick).

20. An antiperspirant product comprising, an aerosol device comprising:
(a) a container comprising a pressurized composition comprising at least one dispersion of cationic colloidal silica particles modified with a multivalent cation, and
(b) at least one propellant and a device for distributing the antiperspirant composition.

21. The antiperspirant composition according to claim 1, further comprising at least one additional antiperspirant active agent.

22. The antiperspirant composition according to claim 1, further comprising at least one additional deodorant active agent.

23. The antiperspirant composition according to claim 1, further comprising at least one oil chosen from volatile and non-volatile, silicone and hydrocarbon-based emollient oils.

24. The antiperspirant composition according to claim 1, further comprising at least one suspension agent.

25. The antiperspirant composition according to claim 1, further comprising at least one organic powder.

26. The antiperspirant composition according to claim 1, further comprising at least one water-immiscible organic liquid phase and at least one agent for structuring said phase.

27. A cosmetic process for treating human perspiration, comprising:
applying to the surface of the skin an effective amount of a dispersion of cationic colloidal silica particles,
wherein the dispersion of cationic colloidal silica particles are modified with a multivalent cation.

28. The cosmetic process according to claim 27, wherein the antiperspirant composition is conditioned such that it can be contained in an aerosol device, a pump-dispenser bottle, a roll-on, a device equipped with a perforated wall, or a wand (stick).

29. A cosmetic process for treating body odor, comprising:
applying to the surface of the skin an effective amount of a dispersion of cationic colloidal silica particles modified with a multivalent cation.

30. The cosmetic process according to claim 29, wherein the antiperspirant composition is conditioned such that it can be contained in an aerosol device, a pump-dispenser bottle, a roll-on, a device equipped with a perforated wall, or a wand (stick).
